(19) United States
(12) Patent Application Publication
(10) Pub. No.: US 2008/0281343 A1
(43) Pub. Date: Nov. 13, 2008

(54) Surgical tool

(75) Inventors: Christopher C. Dewey, Oldwick, NJ (US); Amardeep Singh Dugal, Davie, FL (US); Rony Abovitz, Hollywood, FL (US)

Correspondence Address: FAY SHARPE LLP, 1100 SUPERIOR AVENUE, SEVENTH FLOOR, CLEVELAND, OH, 44114, US

(73) Assignee: MAKO SURGICAL CORP.

(21) Appl. No.: 11/807,018

(22) Filed: May 25, 2007

Related U.S. Application Data
(60) Provisional application No. 60/809,519, filed on May 30, 2006.

(51) Int. Cl. A61B 17/32

(52) U.S. Cl. 606/180

(57) ABSTRACT

A surgical tool (10) includes a cutting element (20), a first portion (30, 60; 40, 70) configured to supply a fluid to a surgical site, and a second portion (40, 70; 30, 60) configured to apply a pressure to the surgical site. The first portion, the cutting element, and the second portion are integrated as a single component.

### CROSS REFERENCE TO RELATED APPLICATIONS

This application claims priority from U.S. Provisional Patent Application Ser. No. 60/809,519, filed May 30, 2006, which is hereby incorporated by reference herein in its entirety.

### BACKGROUND OF THE INVENTION

1. Field of the Invention

The invention relates to a surgical tool and, more particularly, to a surgical tool for cutting the anatomy of a patient.

2. Description of Related Art

Conventional cutting tools may be used to cut and/or sculpt a patient's anatomy during a surgical procedure. For example, during a knee replacement procedure, a surgeon may use a surgical burr to sculpt a tibia T and a femur F to receive a prosthetic device, such as a unicondylar knee implant 500 (shown in FIG. 6) that includes a femoral component 502 and a tibial component 504. In addition to cutting bone, a surgeon may use conventional cutting tools to cut soft tissue, such as cartilage and ligaments.

One drawback of conventional cutting tools is that the rotating or oscillating cutting element of the cutting tool may impinge upon portions of the anatomy that are not intended to be cut. This may occur, for example, when a surgeon operates a conventional cutting tool in a confined anatomical space, such as during minimally invasive surgery (MIS). MIS involves the performance of surgery through incisions that are considerably smaller than incisions used in traditional surgical approaches. The small incision size reduces the surgeon's ability to view and access the anatomy and creates a confined surgical workspace. For example, in minimally invasive orthopedic joint replacement, limited visibility and limited access to the joint increase the complexity of the bone sculpting procedure and the risk that surrounding anatomy will sustain damage from unintended impingement of the cutting tool.

Another drawback of conventional cutting tools is that during a cutting operation, additional tools may be required. For example, a surgeon typically needs to introduce an irrigation tool to provide fluid to the surgical site to flush and lubricate the site. Similarly, the surgeon may need to introduce a suction tool to remove fluid and debris from the surgical site. The need for multiple separate systems to perform cutting, lubrication, and debris removal increases the complexity of the surgical procedure. For example, the simultaneous introduction of multiple tools to the surgical site crowds the surgical site, which increases the risk of damage to surrounding anatomy. In the event the surgical site is too confined to receive more than one tool at a time, the surgeon must stop cutting and remove the cutting tool before he can lubricate or remove debris from the surgical site. As a result, the length of time required to perform the surgical procedure is increased. Further, multiple separate systems may occupy a large amount of space in the operating room and require the use of additional support personnel to operate such systems.

In view of the foregoing, a need exists for a surgical tool that can reduce the risk of damage to the anatomy in the vicinity of the surgical site, reduce the number of tools required to perform a surgical procedure, and enable a surgeon to perform the functions irrigation and/or debris removal without having to stop the cutting procedure and/or remove the cutting tool from the surgical site.

### SUMMARY OF THE INVENTION

An aspect of the present invention relates to a surgical tool. The surgical tool includes a cutting element, a first portion configured to supply a fluid to a surgical site, and a second portion configured to apply a pressure to the surgical site. The first portion, second portion, and cutting element are integrated as a single component.

Another aspect of the present invention relates to a surgical tool. The surgical tool includes a cutting element mounted to a rotatable drive shaft and a tubular outer portion surrounding the cutting element and drive shaft. The outer tubular portion and the drive shaft are configured for relative rotational and axial movement relative to each other.

Yet another aspect of the present invention relates to a surgical method. The surgical method includes introducing a surgical tool to a surgical site, cutting a portion of a bone of a patient with the surgical tool, irrigating the surgical site with the surgical tool, and removing debris from the surgical site with the surgical too. The steps of cutting, irrication and removing debris may be performed simultaneously.

Yet another aspect of the present invention relates to a surgical tool. The surgical tool includes a hollow drive shaft, a motor configured to rotate the hollow drive shaft, a rotary cutting element disposed at one end of the hollow drive shaft, and a tubular outer element surrounding the hollow drive shaft and at least a portion of the rotary cutting element. The surgical tool also includes a bearing mechanism and a means for moving the hollow drive shaft and the tubular outer element axially relative to each other to move the rotary cutting element between a retracted position within the tubular outer element and an extended position in which the rotary cutting element extends beyond an end of the tubular outer element. The surgical tool further includes a source of irrigation fluid connected with the hollow drive shaft or the tubular outer element to supply irrigation fluid adjacent the rotary cutting element and a source of suction connected with the hollow drive shaft or the tubular outer element to withdraw fluids and bone chips from adjacent the rotary cutting element.

### BRIEF DESCRIPTION OF THE DRAWINGS

The accompanying drawings, which are incorporated in and constitute a part of this specification, illustrate embodiments of the invention and together with the description serve to explain principles of the invention.

FIG. 1 is a perspective view of an embodiment of a surgical tool according to the present invention.

FIG. 2 is a perspective view of a distal end of the surgical tool of FIG. 1.

FIG. 3 is a perspective view of a proximal end of the surgical tool of FIG. 1.

FIG. 4 is a side view of the surgical tool of FIG. 1 with a cutting element in a retracted position.

FIG. 5 is a side view of the surgical tool of FIG. 1 with the cutting element in an extended position.

FIG. 6 is a perspective view of a unicondylar knee implant in a knee joint.

FIG. 7 is a perspective view of the surgical tool of FIG. 1 being used to sculpt a femur.

FIG. 8 is a perspective view of the surgical tool of FIG. 1 being used to sculpt a tibia.

FIG. 9 is a block diagram of an embodiment of a surgical method according to the present invention.

FIG. 10 is a plan view of a shape to be sculpted in a bone surface according to an embodiment of the present invention.

FIG. 11 is a plan view of a shape to be sculpted in a bone surface according to an embodiment of the present invention.

FIG. 12 is a side view of a the surgical tool of FIG. 1 showing irrigation and vacuum systems.

### DETAILED DESCRIPTION OF PREFERRED EMBODIMENTS

Presently preferred embodiments of the invention are illustrated in the drawings. Although this specification refers primarily to cutting bone, it should be understood that the subject matter described herein is applicable to other parts of the body, such as, for example, soft tissue, ligaments, cartilage, organs, and muscles.

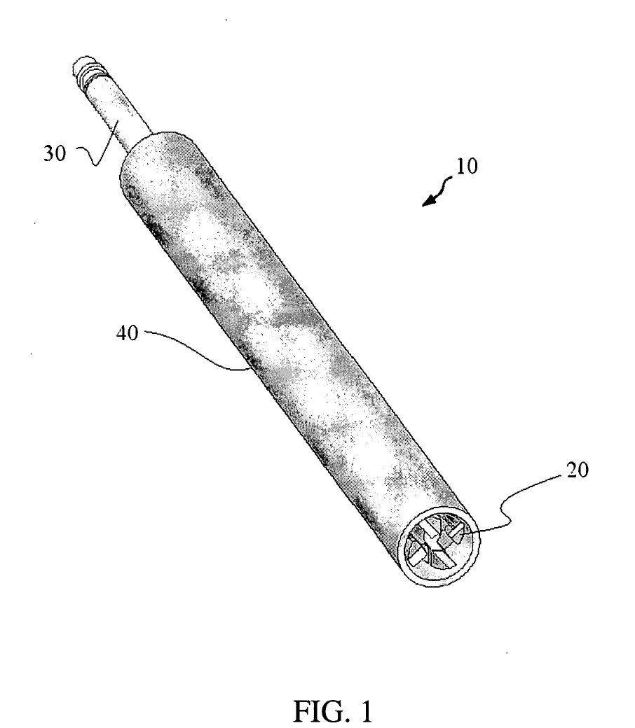

FIGS. 1 to 3 show an embodiment of a surgical tool 10 according to the present invention. The surgical tool 10 comprises a cutting element 20, an inner portion 30, and an outer portion 40.

The cutting element 20 of the surgical tool 10 is configured to cut and/or sculpt an anatomy of a patient, such as bone or soft tissue. The cutting element 20 may include any conventional cutting element. For example, the cutting element 20 may comprise a blade or blades, a spherical burr, or the like and may include cutting surfaces on the bottom and/or the sides of the cutting element 20. The cutting element may be adapted to oscillate but is preferably a rotary cutting element as shown in FIG. 1. The cutting element 20 may be made of any conventional material suitable for use in a surgical cutting instrument, such as, for example, 300 and 400 series stainless steels, 17-4 and 17-7PH stainless steels, zirconia ceramin, nitinol, titanium, and the like.

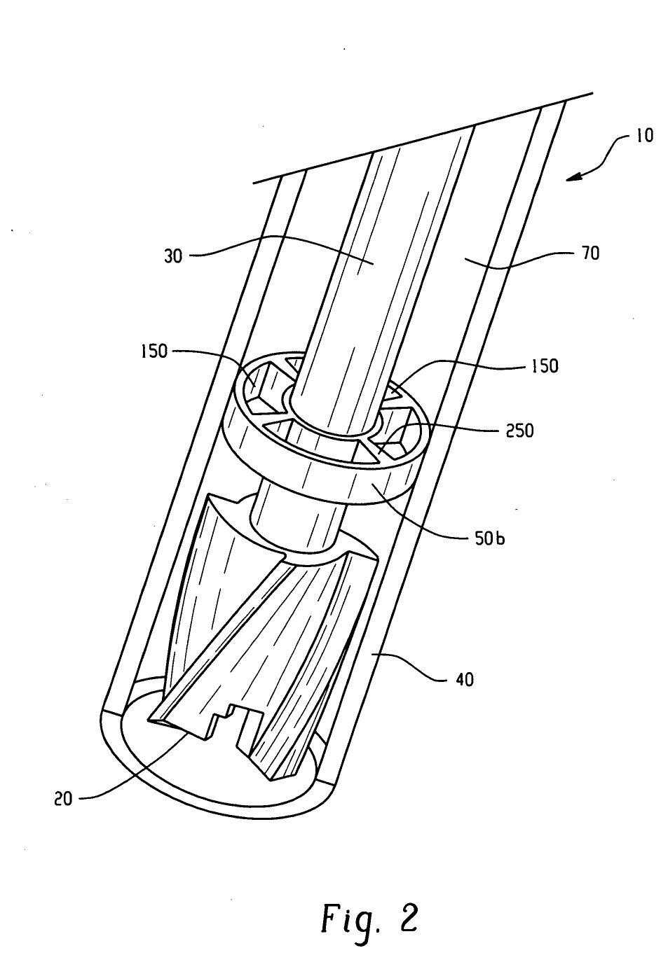

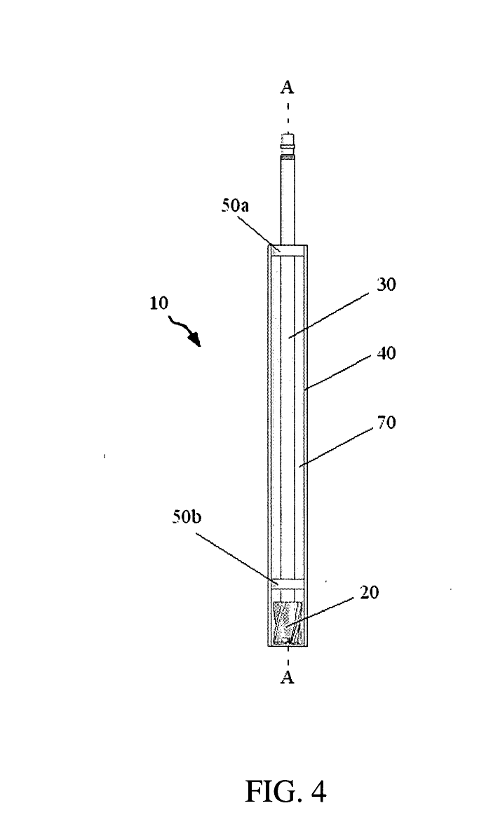

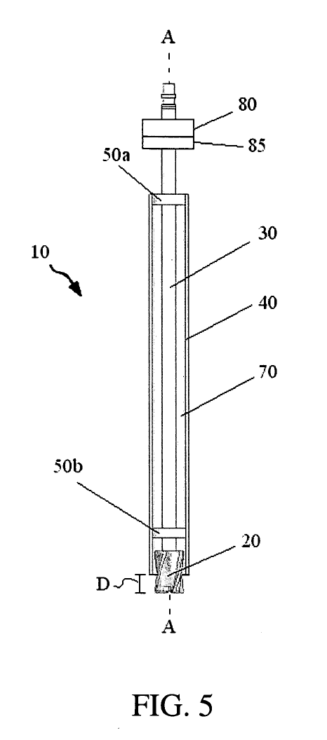

In one embodiment, the cutting element 20 is configured to be disposed within the outer portion 40 of the surgical tool 10 and to be controllably extended therefrom. For example, the cutting element 20 may be adapted to be axially moveable along an axis A-A between at least a first position and a second position, such as a retracted position (shown in FIG. 4) and an extended position (shown in FIG. 5). In the retracted position (shown in FIG. 4), the cutting element 20 is fully enclosed or encapsulated by the outer portion 40 of the surgical tool 10. One advantage of encapsulating the cutting element 20 within the outer portion 40 is that the outer portion 40 effectively functions as a protective casing or guard that prevents the cutting element 20 from contacting portions of the anatomy that are not intended to be cut. For example, when the surgeon is guiding the surgical tool 10 to the surgical site or withdrawing the surgical tool 10 from the patient's body, he can move the cutting element 20 to the retracted position to avoid inadvertently contacting and damaging sensitive anatomy with the cutting element 20. As illustrated in FIGS. 2, 4, and 5, the outer portion 40 may be opaque or transparent to enable the surgeon to see into the surgical tool 10, for example, to view the position of the cutting element 20 or to determine whether debris is clogging the tool 10.

In the extended position (shown in FIG. 5), the cutting element 20 may be disposed in any position from partially exposed (i.e., at lest a portion of the cutting element 20 extends beyond an end of the outer portion 40 of the surgical tool 10) to fully exposed (i.e., the entire cutting element 20 extends beyond the outer portion 40 of the surgical tool 10). In one embodiment, the cutting element 20 is configured to extend, relative to the outer portion 40, one or more prescribed increments, such as one or more selectable distances D. As shown in FIG. 5, the selectable distance D may be measured from a distal end of the outer portion 40. Preferably, the selectable distance D corresponds to a desired cutting depth and may be selected by the operator of the tool 10. For example, if the surgeon desires to cut a bone surface to a depth of 1 mm, the selectable distance D can be set to 1 mm, and the cutting element 20 can be moved axially along the axis A-A until the selectable distance D is reached. In this manner, the cutting element 20 may be set to an operator selected increment or distance from the end of the second portion 40 (e.g., 1 mm, 1.5 mm, 2 mm, 2.5 mm, etc.).

To enable the cutting element 20 to move axially along the axis A-A, the surgical tool 10 may include a means for axially displacing the cutting element 20, such as a mechanism 80 as shown in FIG. 5. The mechanism 80 may be any known device for moving a tool tip axially, such as, for example, a simple mechanical depth selection switch or a more sophisticated microprocessor controlled electrical stepper motor. Additionally, the surgical tool 10 may include a biasing element 85 configured to bias the cutting element 20 toward the retracted position so that the cutting element 20 is in a “safe” configuration. In the safe configuration, the cutting element 20 is enclosed by the outer portion 40 of the surgical tool 10 and cannot impinge upon the patient's anatomy. The biasing element may be, for example, a simple electromechanical cut-off safety switch/mechanism.

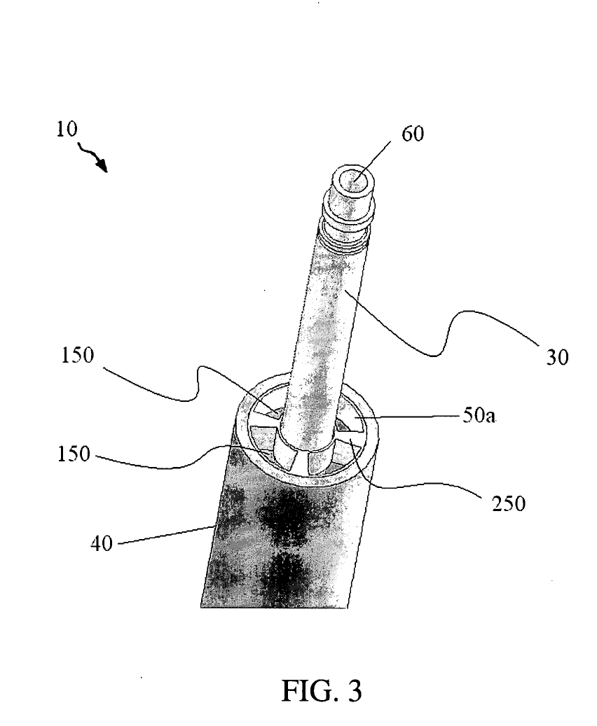

In addition to cutting, the surgical tool 10 is configured to perform irrigation and vacuum (or debris removal) functions. For example, the surgical tool 10 may include a first portion configured to perform an irrigation function by supplying fluid to the surgical site to flush and lubricate the surgical site. The surgical tool 10 may also include a second portion configured to perform a vacuum function by applying a pressure (e.g., a negative or suction pressure) to the surgical site for removal of debris (e.g., bone chips, cutting debris, etc.) and fluid (e.g., flushing fluid, blood, etc.). The pressure applied by the second portion may also be a positive pressure, such as a flow of air to dry the surgical site. In one embodiment, the first portion (which supplies the fluid) may be the outer portion 40 of the surgical tool 10, and the second portion (which applies the pressure) may be the inner portion 30. To enable delivery of fluid and the application of pressure, both the inner portion 30 and the outer portion 40 are hollow. For example, the inner portion 30 may include a passage 60 (shown in FIG. 3), and the outer portion 40 may include a passage 70 (shown in FIG. 5). In a preferred embodiment, the first portion (which supplies the fluid) is the inner portion 30 of the surgical tool 10, and the second portion (which applies the pressure) is the outer portion 40. Although the present invention contemplates either embodiment, the preferred embodiment is described below.

As shown in FIGS. 1-3, the cutting element 20, the inner portion 30, and the outer portion 40 of the surgical tool 10 are integrated as a single component. For example, the cutting element 20 may be supported by the inner portion 30, and the inner portion 30 may be fitted concentrically into the outer portion 40 so that at least a portion of the inner portion 30 is disposed within the outer portion 40. In a preferred embodiment, the inner portion 30 of the surgical tool 10 comprises a hollow drive shaft that supports the cutting element 20 and transmits a rotational driving force to the cutting element 20. As shown in FIG. 3, the hollow inner portion 30 includes the passage 60 through which fluid (or pressure) can be introduced to the surgical site. Similarly, the outer portion 40 comprises a surrounding casing (e.g., a tubular casing or hollow tube) that rotationally receives the inner portion 30 and the cutting element 20. As shown in FIG. 2, the passage 70 exists between an internal surface of the outer portion 40 and an external surface of the inner portion 30. The passage 70 enables the application of pressure (or the introduction of fluid) to the surgical site. When the pressure is negative (i.e., a suction pressure), cutting debris and fluids are extracted from the surgical site via the passage 70.

Because the cutting element 20, the inner portion 30, and the outer portion 40 are integrated into a single component, the surgical tool 10 may be used for cutting, irrigation, and/or debris removal, which reduces the number of separate surgical tools required to perform a surgical cutting operation. Additionally, incorporating the functions of cutting, irrigation, and debris removal into a single tool (a) enables each of the functions to be performed independently (i.e., one function at a time) or in combination (i.e., two or more functions performed simultaneously) and (b) eliminates the need to change out surgical tools between functions. As a result, the time to perform a surgical cutting operation is reduced.

The surgical tool 10 includes at least one element configured to stabilize the cutting element 20 and/or the inner portion 30 on which the cutting element 20 is disposed. For example, the surgical tool 10 may include one or more stabilization elements disposed between the inner portion 30 and the outer portion 40. The number of stabilization elements depends on the design of the surgical tool 10. For example, as a length of the surgical tool 10 increases the number of stabilization elements needed will also increase. In one embodiment, as shown in FIGS. 2 and 3, the stabilization element may include a spacer 50a located in the passage 70 near a proximal end of the outer portion 40 and a spacer 50b located in the passage 70 in a vicinity of the cutting element 20. The spacers 50a and 50b axially stabilize the inner portion 30 and thus the cutting element 20 that is coupled to the inner portion 30. The spacers 50a and 50b may be affixed to the inner portion 30 and/or the outer portion 40 in any known manner, such as with an interference fit or adhesive. As shown in FIGS. 2 and 3, the spacers 50a and 50b each include one or more apertures 150 adapted to enable particulate matter (e.g., bone debris, fluid, etc.) to traverse the spacers 50a and 50b as the particulate matter is vacuumed away from the surgical site. The spacers 50a and 50b may also include vanes 250 disposed between the apertures 150. The vanes 250 may be straight (as shown in FIGS. 2 and 3) or angled. One advantage of angling the vanes 250 is that the vanes 250 are then able to function as impellers to pump debris and fluid away from the cutting element 20 more effectively.

Each of the inner portion 30, the outer portion 40, and the spacers 50 may be made of any material suitable for use in a surgical instrument. Suitable materials include, for example, stainless steel, aluminum, titanium, and the like. In one embodiment, the outer portion 40 is made of a translucent material, such as a polycarbonate resin thermoplastic. Use of a translucent material is advantageous because the surgeon can see into the passage 70 to determine whether debris is clogging the passage 70 and/or the apertures 150 of the spacers 50a and 50b. In the event the passage 70 and/or the apertures 150 become clogged, the surgeon may use alternate cycles of suction and irrigation to unclog the passage 70 and/or the apertures 150.

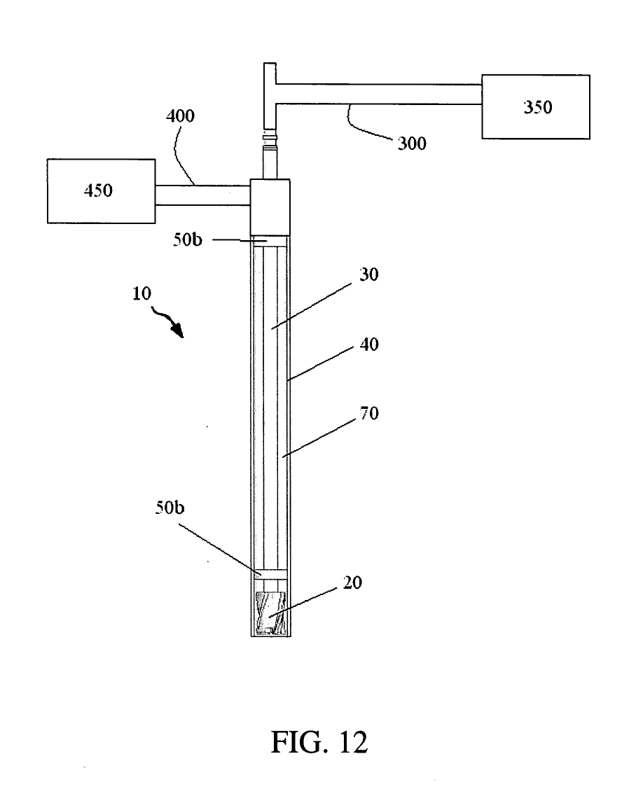

The surgical tool 10 additionally includes irrigation and vacuum components. As is well known, the irrigation components may comprise irrigation lines 300 and an irrigation pump 350, and the vacuum components may comprise vacuum lines 400 and a vacuum pump 450. As shown in FIG. 12, the irrigation lines 300 may be connected (e.g., either directly or via connectors) to the passage 60 to transmit fluid from the irrigation pump 350 to the passage 60. In this manner, fluid may be delivered to the surgical site to flush and lubricate the surgical site. Similarly, the vacuum lines 400 may be connected (e.g., either directly or via connectors) to the passage 70 to apply a vacuum generated by the vacuum pump 450 to the passage 70. In this manner, suction may be applied to the surgical site to remove debris and fluid from the surgical site. As is well known, the surgical tool 10 may also include a motor for driving the cutting element 20 and a power line for supplying electrical (or pneumatic) power to the motor. The motor may be any conventional motor suitable for driving a rotary surgical tool, such as a motor for driving a spherical burr.

The surgical tool 10 is adapted to be connected to a power source to provide power for the cutting element drive motor and the irrigation and vacuum pumps. The power source may be any known power source, such as, for example, an electrical outlet, a battery, a fuel cell, and/or a generator and may be connected to the surgical system 10 using conventional hardware (e.g., cords, cables, surge protectors, switches, battery backup/UPS, isolation transformer, etc.). The surgical tool 10 may also include additional components such as a controller and a user input device for controlling the cutting element 20, the irrigation components, and/or the vacuum components. For example, the user input device may be a foot pedal (or other switching device) that can be positioned on the floor of the operating room in proximity to the surgeon. Depressing the foot pedal causes the power source to supply power to the cutting element 20 (or to a compressed air supply in the case of a pneumatic cutting element), the irrigation pump, and/or the vacuum pump. Conversely, releasing the foot pedal disrupts the flow of power to the cutting element 20, the irrigation pump, and/or the vacuum pump.

To activate the surgical tool 10 for a cutting operation, the surgeon turns the surgical tool 10 ON (e.g., by depressing the foot pedal). In one embodiment, when the surgeon turns the surgical tool 10 ON, power is applied to the cutting element drive motor and to the mechanism 80 so that rotation of the cutting element 20 and extension of the cutting element 20 out of the outer portion 40 begin simultaneously. Alternatively, the rotation and extension may begin one after the other. To deactivate the surgical tool 10, the surgeon turns the surgical tool 10 OFF (e.g., by depressing the foot pedal). In one embodiment, when the surgeon turns the surgical tool 10 OFF, the cutting element 20 shuts off and begins retracting into the outer portion 40 of the surgical tool 10 simultaneously. Alternatively, the shut off and retraction may begin one after the other. One advantage of retracting the cutting element 20 at the same time the cutting element 20 is shut off is that even though the cutting element 20 may still be rotating (e.g., due to momentum), the cutting element 20 is effectively disabled because the sharp portions of the cutting element 20 are retracted and thus enclosed by the outer portion 40 so that there is no possibility of contact with the anatomy. Thus, the surgical tool 10 is in the safe configuration. As a result, the surgeon can irrigate the surgical site, remove debris from the surgical site, and/or withdraw the surgical tool 10 from the patient's body without fear of inadvertently contacting (and possibly damaging) the patient's anatomy with the cutting element 20.

In a preferred embodiment, shut off and/or retraction of the cutting element 20 is incorporated as an automatic safety feature of the surgical tool 10. For example, the surgical tool 10 may include a controller (as is well known) adapted to shut off the cutting element 20 (e.g., by disrupting a flow of power to surgical tool 10) in the event of a system fault or other adverse condition. In one embodiment, the cutting element 20 is an electric tool and includes a relay disposed along an electrical connection between the foot pedal (or other user input device) and the cutting element drive motor. The relay is closed under normal operating conditions so that the cutting element 20 is activated when the surgeon depresses the foot pedal. If a fault is detected, however, the controller commands the relay to open so that the cutting element 20 cannot be activated even if the surgeon depresses the foot pedal. In the case of a pneumatic cutting element 20, a pneumatic shutoff valve may be disposed in an air connection between the foot pedal and the cutting element drive motor. When the fault or other adverse condition is removed, the controller may issue a signal, such as a “fault cleared” signal, so that the surgeon may initiate operation of the cutting element 20 and resume the cutting procedure. The safety feature of the surgical tool 10 may also be activated by a surgical system with which the surgical tool 10 is being used, such as, for example, a robotic surgical system as disclosed in U.S. patent application Ser. No. 11/357,197, U.S. Pub. No. 2006/0142657, filed Feb. 21, 2006, and incorporated by reference herein in its entirety. In a preferred embodiment, the robotic surgical system is the HAPTIC GUIDANCE SYSTEM™ available from MAKO SURGICAL CORP.® in Ft. Lauderdale, Fla.

In addition to shutting off the cutting element 20, an additional precaution may include automatically moving the cutting element 20 into the retracted position when a system fault or other adverse condition is detected. As discussed above, retraction may be accomplished by activating the biasing element 85 which is configured to bias the cutting element 20 in the retracted position. When the cutting element 20 is retracted, the surgical tool 10 is in the safe configuration. Thus, the surgeon can safely withdraw the surgical tool 10 from the patient's body so that he can investigate and/or correct the system fault or other adverse condition. The surgical tool controller may be programmed to trigger a fault when an undesirable condition arises, such as, for example, a power failure or system problem (e.g., motor overheating or failure). During a surgical navigation procedure that incorporates a tracking system (e.g., an optical camera) to track objects (e.g., the surgical tool 10 and the anatomy), a fault may be triggered, for example, when the tracking system is unable to detect a tracked object (e.g., a tracking array is occluded with blood, a line of site between the camera and a tracking array is blocked, etc.), when the anatomy moves suddenly, and/or when the surgical tool 10 is in an undesirable location relative to the anatomy or a surgical plan. The surgical tool 10 may also include a failsafe configuration where positive activation (e.g., depressing a detent) is required to extend the cutting element 20 against the force of the biasing element 85. One advantage of the failsafe configuration is that the biasing element 85 will return the cutting element 20 to the retracted position even in the absence of a fault signal.

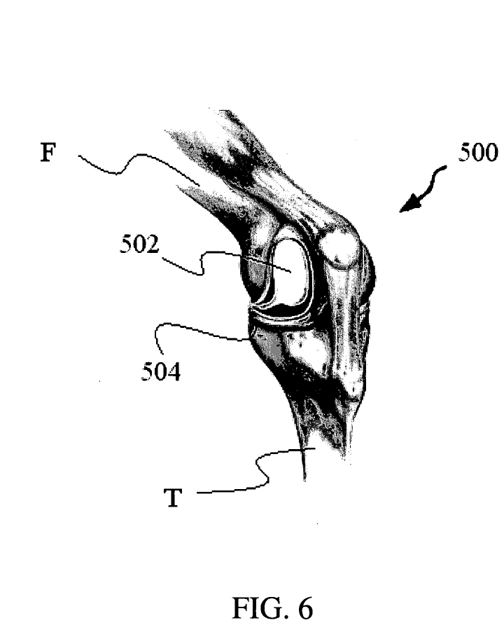

In operation, the surgical tool 10 may be used to perform a surgical cutting operation, to irrigate a surgical site, and/or to remove debris from the surgical site. The surgical tool 10 may be used for a variety of cutting operations but is especially useful for cutting bone. In particular, the surgical tool 10 can be manipulated to sculpt a desired shape into a surface of a bone. For example, the surgeon may operate the surgical tool 10 to prepare the bone to receive a prosthetic device, such as a joint implant. In one embodiment, the surgeon can prepare a femur F and a tibia T of a knee joint to receive components of a unicondylar knee implant 500 (shown in FIG. 6). During bone preparation, the surgeon manipulates the surgical tool 10 to sculpt the femur F to receive the femoral component 502 and the tibia T to receive the tibial component 504. The surgical tool 10 may be manipulated to sculpt each bone so that a shape of the sculpted surface corresponds to a shape of a mating surface of the implant component. Thus, the femur F may be sculpted to have a shape that will mate with the femoral component 502, and the tibia T may be sculpted to have a shape that will mate with the tibial component 504. When sculpting is complete, the components 502 and 504 may be fitted to the bone and secured in place, for example, using bone cement. At anytime during the cutting operation, the surgeon can activate the surgical tool 10 to irrigate and/or remove debris from the surgical site.

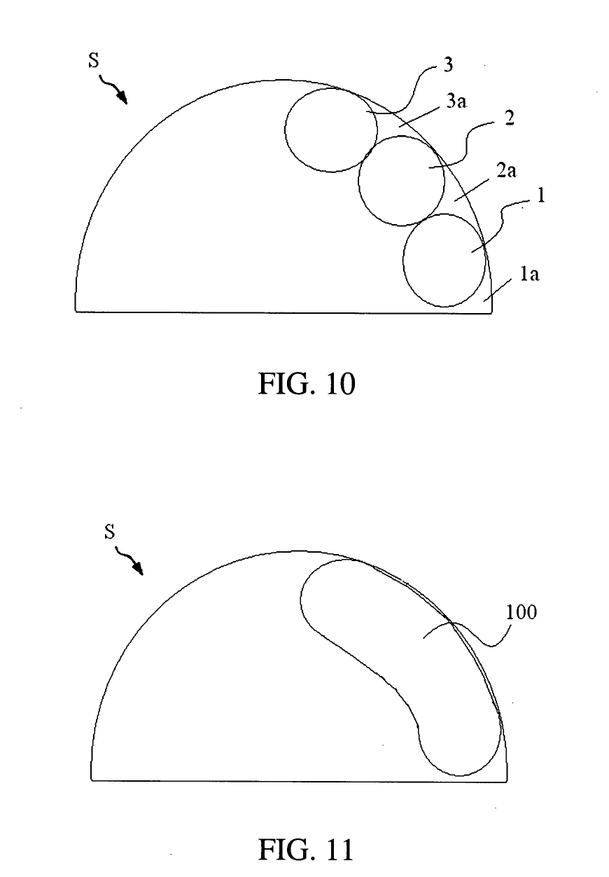

According to one embodiment, as shown in FIGS. 10 and 11, the surgeon can manipulate the surgical tool 10 to sculpt a shape S into a bone, such as the tibia T of the knee joint. The shape S may correspond, for example, to a shape of a corresponding mating surface of the tibial component 504 shown in FIG. 6. In one embodiment, the shape S may be formed by making a series of independent cuts (as illustrated in FIG. 10) with the surgical tool 10. For example, in one embodiment, the shape S may be sculpted by (a) extending the cutting element 20 to a desired cutting depth, (b) activating (e.g., turning on) the cutting element 20 so that the cutting element 20 is rotating, (c) moving the surgical tool 10 to a first position and pressing the exposed portion of the cutting element 20 into the bone to make a first independent cut 1, (d) withdrawing the cutting element 20 from the first cut 1, (e) moving the surgical tool 10 to a second position and pressing the exposed portion of the cutting element 20 into the bone to make a second independent cut 2, (f) withdrawing the cutting element 20 from the second cut 2, (g) moving the surgical tool 10 to a third position and pressing the exposed portion of the cutting element 20 into the bone to make a third independent cut 3, (h) withdrawing the cutting element from the third cut 3, and so on until the desired shape S has been substantially formed. In this embodiment, the shape S could be formed using approximately fourteen independent cuts. Any remaining unwanted bone sections (e.g., sections 1a, 2a, and 3a) can be shaved off of the bone by sweeping the rotating, extended cutting element 20 over the unwanted bone sections.

Alternatively, as opposed to pressing the rotating, extended cutting element 20 into the bone to make the independent cuts 1, 2, and 3, the surgeon may begin each independent cut with the cutting element 20 in the retracted position. In this embodiment, the surgeon (a) moves the surgical tool 10 to the first position and contacts the bone with the distal end of the outer portion 40, (b) activates the cutting element 20 so that the cutting element 20 is rotating, (c) extends the cutting element 20 from the outer portion 40 of the surgical tool 10 to the desired cutting depth to make the first cut 1, (d) retracts the cutting element 20 to the retracted position to withdraw the cutting element 20 from the first cut 1, (e) repositions the surgical tool 10 in the second position, and (f) repeats the procedure to form the remaining independent cuts. In another embodiment, the shape S may be formed by making one or more sweep cuts (as illustrated in FIG. 11). In this embodiment, the surgeon (a) activates the cutting element 20 so that the cutting element 20 is rotating, (b) extends the cutting element 20 to a desired cutting depth, and (c) sweeps the extended cutting element across the surface of the bone to make one or more sweep cuts 100. The surgeon repeats this process until sculpting of the desired shape S is complete.

One advantage of the surgical tool 10 is that once the surgeon has cut the shape S in the bone, he can place a trial implant component on the prepared surface of the bone to test the fit of the particular implant he plans to implant in the joint. If the joint is too loose when the trial implant component is installed on the prepared bone surface, the shape S has been cut too deeply into the bone. The surgeon can correct the loose joint condition by implanting an implant having a greater thickness than the implant selected during surgical planning. For example, the surgeon can implant a tibial component 504 having a tibial insert that is thicker than the tibial component selected during surgical planning. Conversely, if the joint is too tight when the trial implant component is fitted on the prepared bone surface, the shape S is too shallow. Rather than selecting a new implant component, the surgeon can simply extend the cutting element 20 to a new cutting depth and recut the shape S. For example, if the original cutting depth was 1 mm and the joint is too tight, the surgeon can extend the cutting element to 1.5 mm, for example, and recut the shape S.

Thus, according to an embodiment, a surgical method for preparing a surface of a bone with the surgical tool 10 includes the following steps: (a) extending the cutting element 20 of the surgical tool 10 to a first selectable distance from the outer portion 40; (b) preparing the surface of the bone to receive an implant by removing a portion of the bone with the cutting element 20; (c) installing a trial implant component in the prepared surface of the bone; (d) analyzing the fit of the trial implant in the prepared surface of the bone; (e) removing the trial implant component from the surface of the prepared bone; (f) extending the cutting element 20 to a second selectable distance; and (g) recutting the prepared surface of the bone based on the analysis of the fit of the trial implant. Advantageously, while the surface of the bone is prepared to receive the implant, the outer portion 40 of the surgical tool 10 protects adjacent tissue from unintended impingement by the portion of the cutting element 20 enclosed within the outer portion 40.

The ability of the surgeon to incrementally extend the cutting element 20 is particularly useful when the surgical tool 10 is used in combination with a haptic robotic surgical system as disclosed in the above-referenced U.S. Pub. No. 2006/0142657. In a preferred embodiment, the robotic surgical system is the HAPTIC GUIDANCE SYSTEM™ available from MAKO SURGICAL CORP.® in Ft. Lauderdale, Fla. In this embodiment, as described in U.S. Pub. No. 2006/0142657, the surgeon uses surgical planning software to plan the placement of an implant on the patient's bone by positioning a virtual model of the implant on an image of the patient's bone. The planning software generates a virtual haptic object that defines a virtual cutting boundary (e.g., the desired shape S of the bone cut) corresponding the planned implant placement. During surgery, the image of the bone, the haptic object, and the surgical tool 10 are registered to the patient's physical anatomy, and the surgical tool 10 and the anatomy are tracked by a tracking system. As the surgeon manipulates the surgical tool 10 to sculpt the bone, the robotic surgical system provides haptic guidance to the surgeon to maintain the cutting element 20 of the surgical tool 10 within the virtual cutting boundary defined by the haptic object. After sculpting, a trial implant is fitted to the prepared surface of the bone. If a trial implant indicates that the joint is too tight (i.e., the cut is too shallow), the surgeon knows that he must make a deeper cut. Rather than replanning the placement of the implant component so that the planning software can generate a second haptic object defining a deeper virtual cutting boundary, the present invention advantageously enables the surgeon to simply extend the cutting element 20 of the surgical tool 10 to a new increment and recut the bone surface. As a result, the surgical procedure is simplified and the time required to perform the procedure is reduced.

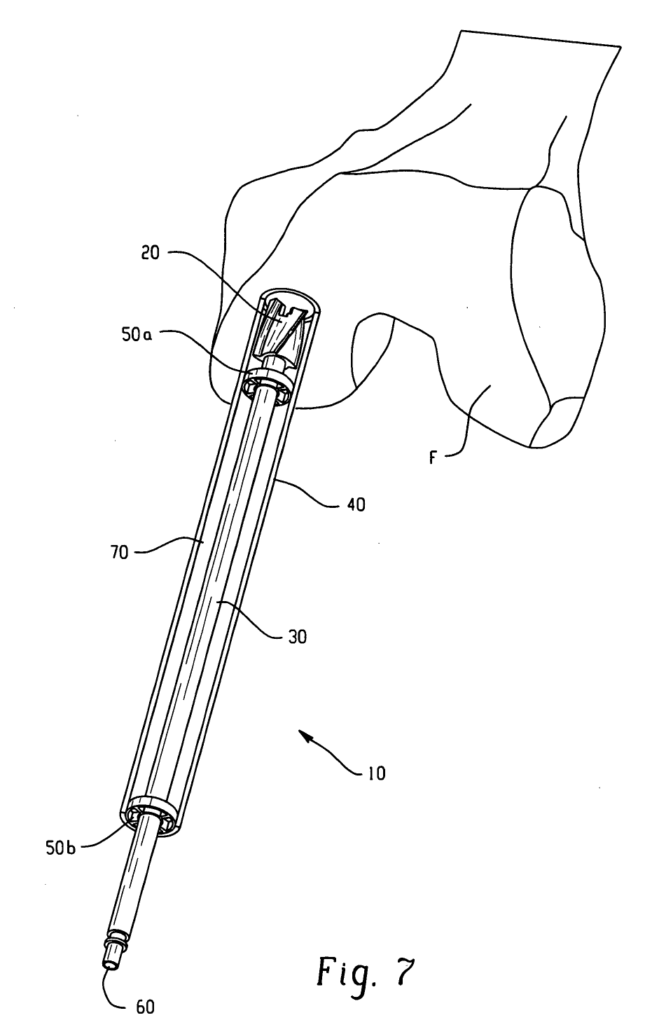

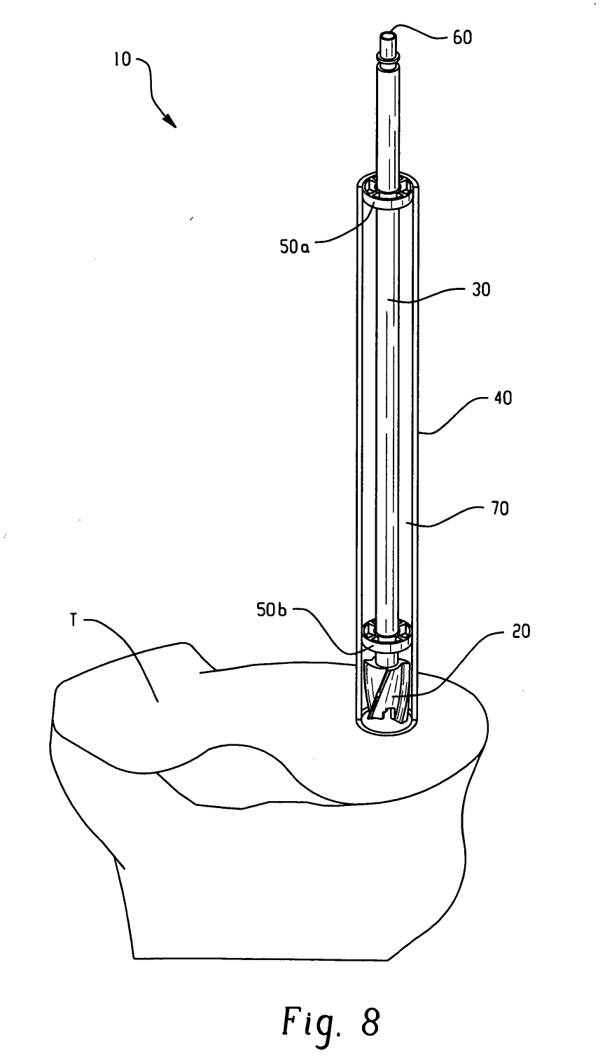

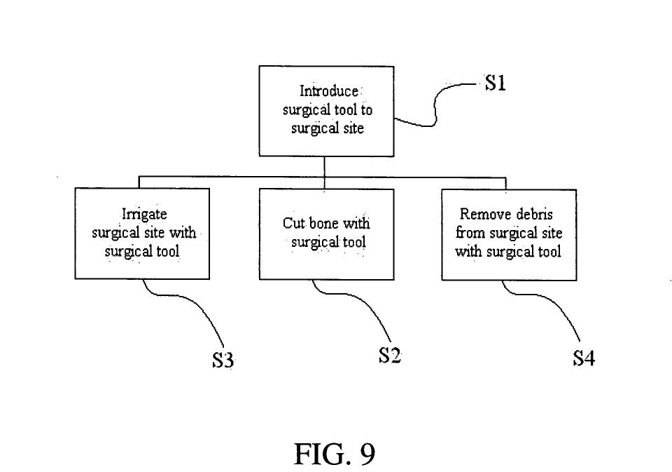

The surgical tool 10 may be used in a variety of surgical procedures to perform a surgical cutting operation, to irrigate a surgical site, and/or to remove debris from the surgical site. One embodiment of a surgical method according to the present invention includes steps S1 to S4, as shown in FIG. 9. In step S1, the surgeon introduces the surgical tool 10 to a surgical site, such as a knee joint of a patient. In step S2, the surgeon cuts a portion of a bone with the surgical tool 10. For example, as shown in FIG. 7, the surgical tool 10 may be used to cut a portion of the femur F. Similarly, as shown in FIG. 8, the surgical tool 10 may be used to cut a portion of the tibia T. In step S3, the surgeon irrigates the surgical site with the surgical tool 10. Irrigation may include, for example, delivering a fluid to the surgical site to flush, lubricate, and/or cool the surgical site. In step S4, the surgeon removes debris from the surgical site with the surgical tool 10. Debris removal may include, for example, applying suction to the surgical site to vacuum away bone debris and fluid. In one embodiment, steps S2, S3, and S4 are performed independently (i.e., separately from one another). In another embodiment, steps S2, S3, and S4 are preformed simultaneously. In another embodiment, any two of the steps S2, S3, and S4 are preformed simultaneously.

The steps of the surgical method may be performed in any order and may include additional steps. For example, in one embodiment, the surgical method includes one or more of the following steps: (a) extending the cutting element 20 from the outer portion 40 of the surgical tool 10; (b) extending the cutting element 20 to a selectable distance (e.g., the selectable distance D shown in FIGS. 4 and 5) relative to the outer portion 40 where the selectable distance corresponds to a desired cutting depth; (c) commencing rotation of the cutting element 20; (d) halting operation of the cutting element 20; (e) retracting the cutting element 20 into the outer portion 40 of the surgical tool 10. The step of halting operation of the cutting element 20 may include terminating operation of the cutting element 20 in response to a signal.

The surgical tool 10 may be used independently or in combination with existing surgical tools and systems, such as, for example, the robotic surgical system disclosed in the above-referenced U.S. Pub. No. 2006/0142657. In a preferred embodiment, the robotic surgical system is the HAPTIC GUIDANCE SYSTEM™ available from MAKO SURGICAL CORP.® in Ft. Lauderdale, Fla. Whether used alone or in combination with other surgical tools and systems, embodiments of the present invention can be advantageously configured to provide a surgical tool that can reduce the time and number of tools required to perform a surgical cutting operation and the risk of unintended impingement of a cutting element on a patient's anatomy.

Other embodiments of the invention will be apparent to those skilled in the art from consideration of the specification and practice of the invention disclosed herein. It is intended that the specification and examples be considered as exemplary only.

What is claimed is:

1. A surgical tool, comprising:
a cutting element,
a first portion configured to supply a fluid to a surgical site, and
a second portion configured to apply a pressure to the surgical site,
wherein the first portion, the second portion, and the cutting element are integrated as a single component.

2. The surgical tool of claim 1, wherein the pressure is a negative pressure.

3. The surgical tool of claim 1, wherein the first portion and the second portion are configured to supply the fluid and apply the pressure simultaneously.

4. The surgical tool of claim 1, wherein the first portion and the second portion are concentric.

5. The surgical tool of claim 1, wherein at least a portion of the first portion is disposed within the second portion.

6. The surgical tool of claim 1, wherein at least a portion of the second portion is disposed within the first portion.

7. The surgical tool of claim 1, wherein one of the first portion and the second portion comprises a hollow shaft which supports the cutting element.

8. The surgical tool of claim 1, wherein the cutting element is configured to be axially moveable between at least a first position and a second position.

9. The surgical tool of claim 8, wherein the cutting element is enclosed by the first portion or the second portion when the cutting element is in the first position.

10. The surgical tool of claim 1, wherein at least a portion of the cutting element extends beyond an end of the first portion or the second portion when the cutting element is in the second position.

11. The surgical tool of claim 1, wherein the cutting element is disposed within one of the first portion and the second portion and is configured to be controllably extended from the one of the first portion and the second portion.

12. The surgical tool of claim 1, wherein the cutting element is configured to move at least one selectable distance.

13. The surgical tool of claim 1, further comprising at least one element configured to stabilize the cutting element.

14. The surgical tool of claim 13, wherein the at least one element is disposed between the first portion and the second portion and includes at least one aperture configured to enable particulate matter to traverse the element.

15. The surgical tool of claim 1, wherein:
the cutting element includes a rotary cutting element;
one of the first and second portions includes a hollow drive shaft which transmits rotational driving force to the rotary cutting element; and
the other of the first and second portions includes a tubular casing in which the rotary cutting element and the hollow drive shaft are rotationally received.

16. The surgical tool of claim 15, further including:
a means for axially displacing the rotary cutting element relative to the tubular casing to extend a portion of the rotary cutting element to a user selected distance from an end of the tubular casing.

17. The surgical tool of claim 15, further including:
a biasing element which biases the rotary cutting element toward a non-extended position in the tubular casing.

18. A surgical tool, comprising:
a rotatable drive shaft;
a cutting element mounted to the drive shaft;
a tubular outer portion surrounding the cutting element and drive shaft, the outer tubular portion and the drive shaft being configured for relative rotational and axial movement relative to each other.

19. The surgical tool of claim 18, further including:
a means for biasing the cutting element to a retracted position within the tubular outer portion; and,
a means for causing relative axial displacement between the cutting element and the tubular outer portion to extend the cutting element relative to the tubular outer portion.

20. The surgical tool of claim 18, wherein the drive shaft is hollow and further including:
a source of irrigating fluid; and,
a source of suction, one of the source of irrigating fluid and the source of suction being fluidly connected with the hollow drive shaft and the other being fluidly connected with the tubular outer portion.

21. A surgical method, comprising:
introducing a surgical tool to a surgical site,
cutting a portion of a bone of a patient with the surgical tool,
irrigating the surgical site with the surgical tool, and
removing debris from the surgical site with the surgical tool,
wherein the steps of cutting, irrigation, and removing debris are performed simultaneously.

22. The surgical method of claim 21, further comprising extending a cutting element of the surgical tool a selectable distance relative to a surrounding casing, wherein the selectable distance corresponds to a desired cutting depth.

23. The surgical method of claim 21, further comprising at least one of retracting a cutting element of the surgical tool into a protective casing and halting operation of the cutting element.

24. The surgical method of claim 21, further comprising at least one of extending a cutting element of the surgical tool from a protective casing and terminating operation of the cutting element in response to a signal.

25. The surgical method of claim 21, further comprising:
extending a cutting element of the surgical tool to a first selectable distance from a surrounding casing,
preparing a surface of the bone to receive an implant by removing a portion of the bone with the cutting element,
installing a trial implant component in the prepared surface of the bone,
analyzing a fit of the trial implant in the prepared surface of the bone,
removing the trail implant component from the prepared surface of the bone,
extending the cutting element to a second selectable distance, and
recutting the prepared surface of the bone based on the analysis of the fit of the trial implant.

26. The surgical method of claim 25, further comprising, during the step of preparing the surface of the bone to receive the implant, protecting adjacent tissue with the casing of the cutting element.

27. A surgical tool, comprising:
a hollow drive shaft;
a motor configured to rotate the hollow drive shaft;
a rotary cutting element disposed at one end of the hollow drive shaft;
a tubular outer element surrounding the hollow drive shaft and at least a portion of the rotary cutting element;
a bearing mechanism;
a means for moving the hollow drive shaft and the tubular outer element axially relative to each other to move the rotary cutting element between a retracted position within the tubular outer element and an extended position in which the rotary cutting element extends beyond an end of the tubular outer element;
a source of irrigation fluid connected with one of the hollow drive shaft and the tubular outer element to supply irrigation fluid adjacent the rotary cutting element;
a source of suction connected with one of the hollow drive shaft and the tubular outer element to withdraw fluids and bone chips from adjacent the rotary cutting element.
